(19) United States
(12) Patent Application Publication
(10) Pub. No.: US 2023/0072768 A1
(43) Pub. Date: Mar. 9, 2023

(54) Minimally Invasive Cosmetic Procedure to Debulk Fat

(71) Applicant: UGlow Face & Body, Scottsdale, AZ (US)

(72) Inventor: Krista KUTAC, Scottsdale, AZ (US)

(73) Assignee: UGlow Face & Body, Scottsdale, AZ (US)

(21) Appl. No.: 17/528,651

(22) Filed: Nov. 17, 2021

Related U.S. Application Data
(60) Provisional application No. 63/241,336, filed on Sep. 7, 2021.

(51) Int. Cl. A61M 1/00; A61K 35/19; A61B 18/04

(52) U.S. Cl. CPC A61M 1/89; A61B 18/04; A61K 35/19; A61M 2202/08

(57) ABSTRACT

A minimally invasive cosmetic procedure for de-bulking fat includes steps of: (a) injecting platelet rich plasma (PRP) into a subject area; followed by (b) heating fat cells in the subject area using radiofrequency; followed by (c) aspirating fat cells in the subject area using liposuction; followed by (d) injecting platelet rich plasma (PRP) into the subject area again. The PRP injecting step (a) may be performed two to seven weeks prior to the heating and liposuction steps (b) and (c); and the PRP injecting step (d) may be performed several days to four weeks after the heating and liposuction steps (b) and (c).

### CROSS-REFERENCE TO RELATED APPLICATIONS

The current application claims priority to U.S Provisional Application Serial No. 63/241,336, filed Sep. 7, 2021, the entire disclosure of which is incorporated herein by reference.

### BACKGROUND

The current disclosure provides a cosmetic solution for body contouring and permanent fat reduction.

### SUMMARY

An aspect of the current disclosure provides a minimally invasive cosmetic procedure for de-bulking fat comprising steps of: (a) injecting platelet rich plasma (PRP) into a subject area; followed by (b) heating fat cells in the subject area using radiofrequency; followed by (c) aspirating fat cells in the subject area using liposuction; followed by (d) injecting platelet rich plasma (PRP) into the subject area again. In a more detailed embodiment, the PRP injecting step (a) is performed two to seven weeks prior to the heating and liposuction steps (b) and (c); and the PRP injecting step (d) is performed several days to four weeks after the heating and liposuction steps (b) and (c). In an embodiment, the liposuction step (c) uses power-assisted liposuction (PAL). In an embodiment, the heating step (b) uses directional RF technology.

### DETAILED DESCRIPTION

Unlike any prior-art procedure for fat reduction, the current disclosure provides a minimally invasive cosmetic procedure that permanently removes fat cells and regenerates healthy, firmer and younger looking skin. “FirmSculpt” is a unique 3-step protocol that incorporates Radio-Frequency Energy, Platelet Rich Plasma (PRP) and Power Assisted Liposuction. The integration of these technologies delivers dramatic results that cannot be achieved by any single or current combination of technologies available today.

The first step is PRP injections into the treatment area both before and after liposuction. PRP not only regenerates tissue, stimulates collagen production and increases blood flow resulting in less down time as compared to other minimally-invasive liposuction procedures, but also leads to firmer and healthier looking skin. This procedure may be performed anywhere from 4 to 6 weeks (e.g., 30 days) prior to and 4 to 6 weeks after liposuction (e.g., 30 days). Considering the well- known regenerative nature of PRP, by injecting the treatment area prior to liposuction, the fibroblast cells begin to stimulate new collagen. Fibroblasts are the most common cells of connective tissue in humans; and, therefore, by injecting the treatment area with PRP prior to liposuction, the skin will begin to thicken and “firm up” which both maximizes the liposuction results and speeds up the healing process.

The second step of the protocol involves radiofrequency (RF) delivered by the In-Mode BodyTite® machine. Utilizing this revolutionary directional RF technology results in three-dimensional tissue remodeling through fat coagulation and volumetric heating. The primary benefit of this step is skin retraction and tightening which ultimately yields the optimal “firming” that patients have been seeking for decades. As the most common post-operative complaint from liposuction patients are in fact related to “sagging skin” or laxity, by incorporating this leading RF technology patients experience tighter and firmer skin with overall superior results than can be achieved through other fat reduction treatments with or without liposuction alone. After the patient is comfortably anesthetized under awake oral sedation and the tumescent solution with local anesthesia infused (mix of saline, lidocaine and epinephrine), the physician uses RF to safely heat the area and “melt” the fat prior to the liposuction (aspiration).

The third and final step of the procedure is the afore-mentioned Power-Assisted Liposuction (PAL). PAL enables the practitioner to target areas of the body with greater precision than traditional liposuction which again leads to more dramatic results. The practitioner permanently removes the majority of the fat cells using variously sized cannulas to aspirate the fat which is then disposed of properly via strict OSHA guidelines. Patients can often observe their early body contouring with both fat reduction from the liposuction and skin tightening from the RF procedure as the physician is literally sculpting the patient’s body.

In summary, “FirmSculpt” is a totally new concept combining multiple non-invasive technologies with liposuction to create a new multimodal approach to treat in a more permanent manner the long-standing problem of skin laxity after both non-invasive and surgical fat removal which we believe will revolutionize the industry.

Exemplary Proprietary Aspects: Specific proprietary is the pre and post regenerative state health of tissue going through conditioning treatment due to regenerative growth factors and angiogenesis pre and post liposuction. Why this proprietary protocol has not been adapted before is because of the exact steps of the procedure. RF/PRP is known as a sole treatment. Fat grafting/PRP is also known as a sole treatment. But PRP prior to the RF and liposuction then PRP post, has not due to most practitioners either specializing in device procedures such as RF or specializing in cosmetic procedures such as liposuction. Further, it has only been recently known that regenerative procedures in any format have been incorporated into clinic treatments in the US.

Scientific Support: This proprietary procedure protocol works based on the documented science studies of each of the individual treatment steps.
PRP, Platelet Rich Plasma, has been widely studied for its regenerative properties for Ortho joints to soft tissue to hair regeneration to dermis regeneration to wound care.
All due to the stimulation of the body’s growth factors:
   i. TGF-B Transforming Growth Factor beta
   ii. bFGF Basic Fibroblast Growth Factors
   iii. PDGFa-b Platelet Derived Growth Factor
   iv. EGF Epidermal Growth Factor
   v. VEGF Vascular Endothelial Growth Factor
   vi. CTGF Connective Tissue Growth Factor

The PRP regenerative process (both prior to and post-liposuction) shows proprietary results in that the treated areas have regenerative tissue and new blood supply to initiate the liposuction and has regenerative and healing properties post liposuction.

This procedure only derives the proprietary results because of the exact protocol steps of PRP, RF, Liposuction, PRP. This is because normally the body sees artificial removal of adipose as unnatural so surrounding tissues post adipose removal do not uniformly regain uniform internal tissue fluid movement of blood flow. So some tissue areas respond better than others.

However, when the dermal areas are treated prior to adipose removal in a regenerative format with PRP and RF the newly acquired angiogenesis of new blood supplies to those areas allows all areas to format uniformly. And this is further assisted with post treatment regenerative PRP which also greatly assists in the uniform healing properties.

Key collaborative studies are listed below. These studies provide the scientific support for our claims. They include studies on PRP promotion of healthier body adipose, proliferation and of PRP regenerative body tissue healing/proliferation.

Lacci KM, Dardik A. Platelet-rich plasma: support for its use in wound healing. Yale J Biol Med. 2010;83(1):1-9.

Xu P, Wu Y, Zhou L, et al. Platelet-rich plasma accelerates skin wound healing by promoting re-epithelialization. Burns Trauma. 2020;8:tkaa028. Published 2020 Aug 14. doi: 10.1093/burnst/tkaa028

Oneto P, Etulain J. PRP in wound healing applications. Platelets. 2021;32(2):189-199. doi:10.1080/09537104.2020.1849605

Badis D, Omar B. The effectiveness of platelet-rich plasma on the skin wound healing process: A comparative experimental study in sheep. Vet World. 2018;11(6):800-808. doi:10.14202/vetworld.2018.800-808

Lacci KM, Dardik A. Platelet-rich plasma: support for its use in wound healing. Yale J Biol Med. 2010;83(1):1-9.

Chicharro-Alcantara D, Rubio-Zaragoza M, Damia-Gimenez E, et al. Platelet Rich Plasma: New Insights for Cutaneous Wound Healing Management. J Funct Biomater. 2018;9(1):10. Published 2018 Jan 18. doi:10.3390/jfb9010010

Li Y, Mou S, Xiao P, et al. Delayed two steps PRP injection strategy for the improvement of fat graft survival with superior angiogenesis. Sci Rep. 2020;10(1):5231. Published 2020 Mar 23. doi:10.1038/s41598-020-61891-6

Serra-Mestre JM, Serra-Renom JM, Martinez L, Almadori A, D’Andrea F. Platelet-rich plasma mixed-fat grafting: a reasonable prosurvival strategy for fat grafts?. Aesthetic Plast Surg. 2014;38(5):1041-1049. doi: 10.1007/s00266-014-0374-7

Modarressi A. Platlet Rich Plasma (PRP) Improves Fat Grafting Outcomes. World J Plast Surg. 2013;2(1):6-13.

Modarressi A. Platlet Rich Plasma (PRP) Improves Fat Grafting Outcomes. World J Plast Surg. 2013;2(1):6-13

Luck J, Smith OJ, Mosahebi A. A Systematic Review of Autologous Platelet-Rich Plasma and Fat Graft Preparation Methods. PlastReconstr Surg Glob Open. 2017;5(12):e1596. Published 2017 Dec 6. doi:10.1097/GOX.0000000000001596

Each of the above-listed articles are incorporated herein by reference.

### Exemplary Protocol - From Consultation to Liposuction

FirmSculpt is a minimally-invasive, awake-liposuction procedure that combines the radiofrequency and fat emulsion technology of BodyTite® and/or FaceTite® (by InMode®), the regenerative, restorative and rejuvenative properties derived from medical-grade Platelet Rich Plasma (PRP) by DrPRPUSA®, and the advanced technology of a Power-Assisted Liposuction system by HK Surgical.

Below is an exemplary protocol:
1. Consultation. The physician will complete a physical assessment to determine candidacy. The necessary health history, patient intake, HIPPA forms are signed by the consult and Center Director. We utilize EMR for every consultation, patient and treatment. The consult/patient is taken into a treatment room, weighed and assessed by the practitioner. The Physician (Primary Practitioner) will make a recommendation for candidacy. If the consult/patient decides to accept the recommendation and move forward with the procedure, the Physician (Primary Practitioner) will determine whether or not a medical clearance is required (based on the health history and current health of the patient). The Center Director will assist in obtaining payment from patient and scheduling the first and second treatments in the treatment protocol.
2. The first treatment/next step is PRP injections in the areas purchased. This treatment will be performed by either a Nurse Practitioner, Registered Nurse or Physician’s Assistant. This step is completed at least two weeks prior to liposuction (as far as 45 days prior to liposuction).
3. The next step is the day of liposuction. The patient will arrive one hour prior to liposuction to sign the applicable “day-of-procedure” documents. The patient is taken to a pre-op room and assessed again by the Physician (Primary Practitioner) and “pre-op’d”. The patient will be given oral pain and anti-anxiety medication prescribed by the Physician (Primary Practitioner).
4. The patient is taken into the liposuction room, the patient gets on the table and the Physician (Primary Practitioner) will begin locally numbing the patient with tumescent solution (a mixture of saline, lidocaine and epinephrine). This process takes 30-45 minutes.
5. Once the patient is numb, the Physician (Primary Practitioner) begins the BodyTite® (or FaceTite®) step. These are hand-held devices that delivers radiofrequency energy under the skin. The radiofrequency energy emulsifies the fat and assists in post-procedure skin retraction. Depending on the size of the area, and size of the patient, this can take 20-40 minutes.
6. The following step is aspiration. This is done with a power-assisted liposuction device that aspirates the emulsified fat cells. The benefit of the fat being emulsified is that there’s less trauma to the blood vessels and surrounding tissue. Each fat cell is connected to blood cells. Our claim that FirmSculpt® has less down time than traditional liposuction and other protocols of awake-liposuction is due to the aspiration of emulsified fat which produces less trauma which results in less swelling and bruising.
7. The patient is then taken to a recovery room, fitted into a compression garment (to be worn for a minimum of two weeks) observed for 45 minutes by a clinician, the released to a caretaker who signs out the patient and is legally responsible for the patient for the next 24 hours.
8. A nurse or doctor follows up with the patient via phone within 24 hours to ensure there are not concerns or complications.
9. The last step of FirmSculpt® takes place one week later. The patient comes in for a one-week follow-up with a nurse. During this follow up, the patient receives their second round of PRP injections. If the PRP injections are not done during this appointment, it can be done up to four weeks post-liposuction.
10. The patient is scheduled for a 3 month and 6 month follow up appointment.

What is claimed is:

1. A minimally invasive cosmetic procedure for de-bulking fat comprising steps of:
A. injecting platelet rich plasma (PRP) into a subject area; followed by
B. heating fat cells in the subject area using radiofrequency; followed by
C. aspirating fat cells in the subject area using liposuction; followed by
D. injecting platelet rich plasma (PRP) into the subject area again.

2. The minimally invasive cosmetic procedure of claim 1, wherein:
the PRP injecting step A is performed two to seven weeks prior to the heating and liposuction steps B and C; and
the PRP injecting step D is performed several days to four weeks after the heating and liposuction steps B and C.

3. The minimally invasive cosmetic procedure of claim 2, wherein the liposuction step C uses power-assisted liposuction (PAL).

4. The minimally invasive cosmetic procedure of claim 3, wherein the heating step B uses directional RF technology.

5. The minimally invasive cosmetic procedure of claim 1, wherein the liposuction step C uses power-assisted liposuction (PAL).

6. The minimally invasive cosmetic procedure of claims 1, wherein the heating step B uses directional RF technology.

7. A kit for performing a minimally invasive cosmetic procedure for debulking fat, comprising:
a directional radio frequency delivery system;
a power-assisted liposuction system; and
instructions for using the directional radio frequency system and the power-assisted liposuction system in combination with platelet rich plasma injections to perform a cosmetic procedure for debulking fat.

8. The kit of claim 7, wherein the instructions instruct a practitioner to perform the following steps:
A. injecting platelet rich plasma (PRP) into a subject area; followed by
B. heating fat cells in the subject area using the directional radio frequency delivery system; followed by
C. aspirating fat cells in the subject area using the power-assisted liposuction system; followed by
D. injecting platelet rich plasma (PRP) into the subject area again.

9. A kit for performing a minimally invasive cosmetic procedure for debulking fat, comprising:
at least one of a directional radio frequency delivery system or a power-assisted liposuction system; and
instructions for using the at least one of the directional radio frequency system or the power-assisted liposuction system in combination with platelet rich plasma injections to perform a cosmetic procedure for debulking fat.

10. The kit of claim 9, wherein the at least one of the directional radio frequency system or the power-assisted liposuction system is a directional radio frequency system and the instructions instruct a practitioner to perform the following steps:
A. injecting platelet rich plasma (PRP) into a subject area; followed by
B. heating fat cells in the subject area using the directional radio frequency delivery system; followed by
C. aspirating fat cells in the subject area using a power-assisted liposuction system; followed by
D. injecting platelet rich plasma (PRP) into the subject area again.

11. The kit of claim 9, wherein the at least one of the directional radio frequency system or the power-assisted liposuction system is a power-assisted liposuction system and the instructions instruct a practitioner to perform the following steps:
A. injecting platelet rich plasma (PRP) into a subject area; followed by
B. heating fat cells in the subject area using a directional radio frequency delivery system; followed by
C. aspirating fat cells in the subject area using the power-assisted liposuction system; followed by
D. injecting platelet rich plasma (PRP) into the subject area again.
